(19) United States
(12) Patent Application Publication
(10) Pub. No.: US 2022/0184162 A1
(43) Pub. Date: Jun. 16, 2022

(54) PREPARATIONS CONTAINING BERRY EXTRACTS FOR USE IN TREATING CANCER

(71) Applicant: Evonik Operations GmbH, Essen (DE)

(72) Inventors: Jean-Luc HERBEAUX, Cascais (PT); Norbert WINDHAB, Hofheim (DE); Christoph BRÜCHER, Eschborn (DE); Anne BENEDIKT, Frankfurt (DE); Maria STEINKE, Höchberg (DE); Jochen BODEM, Geroldshausen (DE); Wolfgang SCHAMEL, Freiburg im Breisgau (DE)

(73) Assignee: Evonik Operations GmbH, Essen (DE)

(21) Appl. No.: 17/598,426

(22) PCT Filed: Mar. 27, 2020
(86) PCT No.: PCT/EP2020/058647

§ 371 Date: Sep. 27, 2021

(30) Foreign Application Priority Data
Mar. 29, 2019 (EP) 19166068.7

(51) Int. Cl. A61K 36/45; A61K 36/185; A61K 31/7048; A61K 35/17; A61K 38/17; A61K 9/00; A61P 35/00

(52) U.S. Cl. CPC A61K 36/45; A61K 36/185; A61K 31/7048; A61P 35/00; A61K 38/1774; A61K 9/0053; A61K 35/17

(57) ABSTRACT

The present invention is related to composition for use in treating or preventing a cancer in a subject, wherein the cancer comprises cancer cells that are CD4+, and wherein the composition comprises one or more of an extract of black currants, an extract of bilberries, and an anthocyanin.

The present invention is related to compositions for use in treating or preventing a cancer in a subject, wherein the cancer comprises cancer cells that are CD4+, and wherein the composition comprises one or more of an extract of black currants, an extract of bilberries, and an anthocyanin.

Anthocyanins are water-soluble vacuolar pigments that may appear red, purple or blue, depending on the surrounding pH-value. Anthocyanins belong to the class of flavonoids, which are synthesized via the phenylpropanoid pathway. They occur in all tissues of higher plants, mostly in flowers and fruits and are derived from anthocyanidins by addition of sugars. Anthocyanins are glycosides of flavylium salts. Each anthocyanin thus comprises three component parts: the hydroxylated core (the aglycone); the saccharide unit; and the counterion. Anthocyanins are naturally occurring pigments present in many flowers and fruit and individual anthocyanins are available commercially as the chloride salts, e.g. from Polyphenols Laboratories AS, Sandnes, Norway. The most frequently occurring anthocyanins in nature are the glycosides of cyanidin, delphinidin, malvidin, pelargonidin, peonidin and petunidin.

It is known that anthocyanins, especially resulting from fruit intake, have a wide range of biological activities, including antioxidant, anti-inflammatory, antimicrobial and anti-carcinogenic activities, improvement of vision, induction of apoptosis, and neuroprotective effects. Particularly suitable fruit sources for the anthocyanins are cherries, bilberries, blueberries, black currants, red currants, grapes, cranberries, strawberries, and apples and vegetables such as red cabbage. Bilberries, in particular Vaccinium myrtillus, and black currants, in particular Ribes nigrum, are especially suitable.

Bilberries contain diverse anthocyanins, including delphinidin and cyanidin glycosides and include several closely related species of the genus Vaccinium, including Vaccinium myrtillus (bilberry), Vaccinium uliginosum (bog bilberry, bog blueberry, bog whortleberry, bog huckleberry, northern bilberry, ground hurts), Vaccinium caespitosum (dwarf bilberry), Vaccinium deliciosum (Cascade bilberry), Vaccinium membranaceum (mountain bilberry, black mountain huckleberry, black huckleberry, twin-leaved huckleberry), Vaccinium ovalifolium (oval-leafed blueberry, oval-leaved bilberry, mountain blueberry, high-bush blueberry).

Dry bilberry fruits of V. myrtillus contain up to 10% of catechin-type tannins, proanthocyanidins, and anthocyanins. The anthocyanins are mainly glucosides, galactosides, or arabinosides of delphinidin, cyanidin, and—to a lesser extent—malvidin, peonidin, and petunidin (cyanidin-3-O-glucoside (C3G), delphinidin-3-O-glucoside (D3G), malvidin-3-O-glucoside (M3G), peonidin-3-O-glucoside and petunidin-3-O-glucoside). Flavonols include quercetin- and kaempferol-glucosides. The fruits also contain other phenolic compounds (e.g., chlorogenic acid, caffeic acid, o-, m-, and p-coumaric acids, and ferulic acid), citric and malic acids, and volatile compounds.

Black currant fruits (R. nigrum) contain high levels of polyphenols, especially anthocyanins, phenolic acid derivatives (both hydroxybenzoic and hydroxycinnamic acids), flavonols (glycosides of myricetin, quercetin, kaempferol, and isorhamnetin), and proanthocyanidins (between 120 and 166 mg/100 g fresh berries). The main anthocyanins are delphinidin-3-O-rutinoside (D3R) and cyanidin-3-O-rutinoside (C3R), but delphinidin- and cyanidin-3-O-glucoside are also found (Gafner, Bilberry—Laboratory Guidance Document 2015, Botanical Adulterants Program).

EP 1443948 A1 relates to a process for preparing a nutritional supplement (nutraceutical) comprising a mixture of anthocyanins from an extract of black currants and bilberries. Anthocyanins were extracted from cakes of fruit skin produced as the waste product in fruit juice pressing from V. myrtillus and R. nigrum. It could be shown that the beneficial effects of individual anthocyanins are enhanced if instead of an individual anthocyanin, a combination of different anthocyanins is administered orally, in particular a combination comprising both mono and disaccharide anthocyanins. It is thought that the synergistic effect arises at least in part from the different solubilities and different uptake profiles of the different anthocyanins.

The immune system protects organisms from infection with layered defenses of increasing specificity. In simple terms, prevent pathogens such as bacteria and viruses from entering the organism. If bacteria or virus breach the physical barriers, which prevent pathogens from entering the organism, the innate immune system provides an immediate, but non-specific response. Innate immune systems are found in all plants and animals. If pathogens successfully evade the innate response, vertebrates possess a second layer of protection, the adaptive immune system, which is activated by the innate response. Here, the immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered.

The innate immunity embraces a cell mediated part, monocytes amongst other leukocytes, and a humoral part, the complement system. Monocytes are present in the bloodstream where they kill invading microorganisms. They also form the progenitor of macrophages since they differentiate when leaving the blood stream into the surrounding tissue. Macrophages are present in all different types of tissues, covering a broad spectrum of tasks including wound healing, tissue homeostasis or the induction of an inflammatory response. Carrying out diverse functions is enabled by the capability of macrophages to switch between two different polarization statuses: the M₁ and the M₂ macrophage phenotype.

M1 and M2 macrophages differ in the tasks they carry out. M1 macrophages are commonly referred to as pro-inflammatory or classically activated macrophages. They release small soluble proteins, called cytokines, like TNF-α, IL-1β and IL-6 that mediate inflammation. These pro-inflammatory cytokines act locally by binding to receptors on nearby epithelial cells as well as systemically after their distribution through the blood stream. M1 macrophages are furthermore involved in matrix degradation and tissue destruction that is necessary to pave the way for incoming leukocytes during the inflammatory event. Moreover, they exhibit a high level of phagocytic activity. M2 macrophages on the contrary are resident in all healthy tissues and do not trigger an inflammation. Their main function is to clear the interstitial environment through phagocytosis. They secrete for instance the immunosuppressive cytokine IL-10 and represent therefore the opposite pole to M1 macrophages. M2 macrophages also contribute to wound healing processes by an enhanced arginase activity. Macrophages can change their physiology and switch back and forth between the two described phenotypes as a reaction to their environment.

When an infectious agent enters the body, it encounters tissue-resident M2 macrophages that are localized under the epithelium. M2 macrophages are phagocytic and capable of clearing minor infections without triggering an inflammation within a few hours. However, if the number of pathogens exceeds the capacity of the tissue resident macrophages, inflammation occurs. The inflammatory response is initiated by the shift of M2 macrophages towards the proinflammatory M1 phenotype after recognition of foreigners or after binding of complement factors. When proteins of the complement system recognize foreign material, they start the cleavage which leads to the formation of soluble C3a that can bind to receptors on M2 macrophages and thus activate them. The shift of the macrophage phenotype from M2 to M1 leads to an inflammatory response through cytokine release that can act, depending on their stability, either in an autocrine, paracrine or endocrine fashion. The three most important cytokines released by M1 macrophages that contribute to the inflammatory response locally and systemically are TNF-α, IL-1β and IL-6.

The adaptive immune system is a subsystem of the overall immune system that is composed of highly specialized, systemic cells and processes that eliminate pathogens or prevent their growth. The adaptive immune response is antigen-specific and creates immunological memory (through memory B cells and memory T cells) after an initial response to a specific pathogen and leads to an enhanced response to subsequent encounters with that pathogen.

The cells of the adaptive immune system are special types of leukocytes, called lymphocytes. B cells and T cells are the major types of lymphocytes and are derived from hematopoietic stem cells in the bone marrow. B cells are involved in the humoral immune response, whereas T cells are involved in cell-mediated immune response. Both B cells and T cells carry receptor molecules that recognize specific targets. T cells recognize a “non-self” target, such as a pathogen, only after antigens have been processed and presented in combination with a “self” receptor called a major histocompatibility complex (MHC) molecule. T cells are characterized by the presence of a T-cell receptor on the cell surface. There are two major subtypes of T cells: killer T cells and helper T cells. In addition, there are regulatory T cells which have a role in modulating immune response.

Cytotoxic T cells (T_{C} cells, killer T cells) destroy virus-infected cells and tumor cells and are also implicated in transplant rejection. These cells are also known as CD8⁺ T cells since they express the CD8 glycoprotein at their surfaces. These cells recognize their targets by binding to antigen associated with MHC class I molecules, which are present on the surface of all nucleated cells. Through IL-10, adenosine, and other molecules secreted by regulatory T cells, the CD8+ cells can be inactivated to an anergic state, which prevents autoimmune diseases.

T helper cells (T_{H} cells) are immune response mediators and play an important role in establishing and maximizing the capabilities of the acquired immune response, including maturation of B cells into plasma cells and memory B cells, and activation of cytotoxic T cells and macrophages. These cells are also known as CD4⁺ T cells because they express the CD4 glycoprotein on their surfaces. Helper T cells express T cell receptors (TCR) that recognize antigen bound to Class II MHC molecules. The activation of a naive helper T cell causes it to release cytokines, which influences the activity of many cell types, including the APC (Antigen-Presenting Cell) that activated it. Helper T cells require a much milder activation stimulus than cytotoxic T cells and can provide extra signals that “help” activate cytotoxic cells.

When naïve or memory T cells encounter foreign antigen along with proper co-stimulation they undergo rapid and extensive clonal expansion. In mammals, this type of proliferation is unique to cells of the adaptive immune system and requires a considerable expenditure of energy and cellular resources. After initial antigenic stimulation the cell size of T cells increases accompanied by a metabolic switch to glycolysis, which is required to support their growth, proliferation, and effector functions. During this metabolic switch process the metabolism of T cells switches from a catabolic metabolism to an anabolic metabolism. By contrast, quiescent T cells (naïve and memory T cells) have a catabolic metabolism where they use glucose, fatty acids, and amino acids for ATP generation through the TCA cycle and oxidative phosphorylation. Growth factor cytokines increase nutrient transporter expression and are important for cell survival, and in their absence, quiescent cells die of progressive atrophy (Pearce, Curr Opin Immunol. 2010 June 22(3): 314-320).

B cells are the major cells involved in the creation of antibodies that circulate in blood plasma and lymph, known as humoral immunity. Additionally, B cells present antigen (classified as professional antigen-presenting cells, APCs) and secrete cytokines. Like T cells, B cells express a unique B cell receptor (BCR), in this case, a membrane-bound antibody molecule. All the BCR of any one clone of B cells recognizes and binds to only one particular antigen (whole pathogens without any need for antigen processing). Each lineage of B cell expresses a different antibody, so the complete set of B cell antigen receptors represent all the antibodies that the body can manufacture.

Autoimmune disease occurs when T cells recognize and react to ‘self’ molecules, that is, molecules produced by the cells of the host. Activation of ‘autoreactive’ T cells by presentation of autoantigens processed by antigen presenting cells (APC) leads to their clonal expansion and migration to the specific tissues, where they induce inflammation and tissue destruction. Dysfunction of the immune system also occurs in some cancers. In particular, cancers in which the immune cells themselves become cancerous often involve abnormal activation and proliferation of immune cells. An example is T helper cell cancers, such as T cell lymphoma. As indicated above, T helper cells are also known as CD4+ T cells and express the marker CD4 on their surface. In such cancers the CD4+ T population is highly activated and proliferative. Accordingly, one indication of the presence of these cancers is an elevated level of CD4+ T cells in the blood of patient, which causes a shift in the normal ratio of CD4+/CD8+ T cells.

There is some clinical evidence for a beneficial effect on inflammatory markers and oxidative stress in connection with anthocyanins. It was shown that anthocyanins inhibit nuclear factor-kB activation in monocytes and reduce plasma concentrations of pro-inflammatory mediators in healthy adults. These data suggest that supplementation with anthocyanins may have a role in the prevention or treatment of chronic inflammatory diseases by inhibition of NF-kB transactivation and deceased plasma concentrations of pro-inflammatory chemokines, cytokines, and inflammatory mediators (Karlsen et al., J. Nutr. 137: 1951-1954, 2007). In diabetic patients it could be demonstrated that anthocyanin supplementation enhances antioxidant capacity and prevents insulin resistance (Li et al. J Nutr 2015;145:742-8). However, the mode of actions of anthocyanins has not been elucidated, yet.

There are some indications in the literature that berries might act as anti-tumor agents from the immunological perspective in tumor-bearing animals and humans. Moreover, the wide spectrum of phytochemicals suggests that they might influence the functions of multiple immune cells and different aspects of cancer immunity. Cancer immune-therapies are showing promise for some types of cancer because they boost T cells' ability to recognize tumor cells—an essential prelude to destruction. Recognition occurs after dendritic cells present antigen, such as tumor antigen, to T cells, generating an adaptive response (Pan et al., J. Berry Res. 8(3): 163-175, 2018). Nevertheless, more information is needed about the effect of the single components contained in berries on the entire spectrum of immunity to use such berries to enhance immune response during cancer prevention and treatment.

The cytokine network is one important physiological target where anthocyanins and their metabolites as well as hydrolyzed forms get in contact with all blood cells, B and T cells and lymphocytes (lymph, blood, lymph nodes and tissues). In this corresponding compartment, they are promoting and expressing cytokine receptors and cytokines, which are secreted. Immune cells will proliferate after activation and secrete specifically or unspecifically upon receptor and receptor-cofactor activation. Moreover, there is a need for a fine-tuned up- and down regulation to balance the immune response to prevent overshooting side effects such as shock or loss of organ function.

Those cytokines are known for their inhibited or exaggerated role in cancer (loss of immune function and Programmed Cell Death response), immune defects or allergies (loss of moderation) and play a critical role in the clinical fate of most pathogenic virus diseases. This interconnection of the cellular cytokine network is the reason why primary clinical markers like fatigue and fever do not correlate with the survival rate of critical infections such as measles and Ebola.

It is therefore crucial during different phases of such diseases to allow specific and basal cytokine network responses and to still allow a specific co-factor selective activation for immune cell proliferation as shown in our experiments.

In this context, it is of primary clinical and nutritive interest to find safe non toxic active food and therapeutic ingredients which interfere to stabilize the healthy phenotype (input-output and response) of the cytokine network. This is especially important before the virus signals and virus proliferation overrule the healthy cytokine network cells by its many diverse evolutionary emerged ways to interfere with the normal metabolism and anabolism.

All cellular inhibitory (acetyl ascorbic acid) or enzyme inhibiting (COX I,II) compounds including steroidal or non-steroidal anti-inflammatory effectors such as leukotriene-modifying drugs globally modify up- or downstream of inflammatory response and show toxicity such as the (e.g. gastric) damaging effects of any prostaglandin synthesis suppression. However, inflammation depends on the release of tissue factors such as histamines, leukotrienes, and prostaglandins. Steroids prevent this production of leukotrienes, via the arachidonic acid pathway, while nonsteroidal anti-inflammatory drugs more selectively prevent it either via the lipoxygenase (LO) or the cyclooxygenase (CO) pathway. In addition to their anti-inflammatory effects, those drugs also retard fibroblastic growth and proliferation which is a toxic effect.

Many modes of actions have been discussed for many nutritional components, such as fish oil and omega-3 fatty acids, however, with low in vitro evidence and mode of action reduction. Here we present a clear mechanism of an effect to moderate state of disease with orally available nutritive components down to the pure substances of the plant extracts. One major difference to the pharmacology of anti-inflammation is that neither COX nor steroidal pathways need to be involved (leucotrien pathway, thrombocytes and steroide receptors).

In the context it was surprisingly found that extracts of black currants and bilberries specifically reduce immune cell activation, and in particular reduce CD4+ cell levels. Results connect this function to anthocyanins and anthocyanin metabolites contained in the berry extract.

The present invention is related to a composition for use in treating or preventing a cancer in a subject, wherein the cancer comprises cancer cells that are CD4+, and wherein the composition comprises one or more of an extract of black currants, an extract of bilberries, and an anthocyanin.

The present invention is also related to an anthocyanin composition for use in treating or preventing a cancer in a subject, wherein the cancer comprises cancer cells that are CD4+, and wherein the anthocyanin comprises one or more anthocyanins, optionally wherein the anthocyanin composition is an extract of black currants and/or an extract of bilberries.

According to the present invention, it is preferred, when the cancer is a T-cell cancer. In a preferred embodiment, the cancer is a T-cell leukemia or a T cell lymphoma, more specifically Sézary syndrome, Hodgkin's lymphoma, dermatopathic lymphadenitis or indolent MCL (mantle cell lymphoma).

The preparation according to the present invention can be applied as a pharmaceutical composition or as nutritional composition, such as in connection with an anthocyanin-rich diet.

More specifically, the invention is related to a composition for treating or preventing a disease or disorder mediated by immune cell activation in a subject by reducing immune cell activation.

In particular, preferably the subjects who can benefit from the treatment of the present invention are those who have an abnormally high level of CD4+ T cells circulating in their blood, which usually also results in an abnormal ratio of CD4+/CD8+ T cells. Normal levels of CD4⁺ and CD8+ in blood sample analysis for human adults and teenagers are usually found within the ranges of 542-1570 cells/μL blood for CD4+ T cells and 310-820 cells/μL blood for CD8+ T cells, the healthy ratio of CD4+/CD8+ being between 1 and 2.3.

Accordingly, in a preferred embodiment a blood sample taken from the subject comprises more than 1200 CD4+ cells/μL, optionally more than 1600 CD4+ cells/μL. Moreover, it is preferred, when a blood sample taken from the subject has a CD4+/CD8+ cell ratio above 2, optionally above 2.5. In a preferred embodiment, the composition reduces the CD4+ cell count and/or the CD4+/CD8+ cell ratio.

In a preferred embodiment, the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and bilberries.

It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%.

It is preferred, according to the present invention, when the extract is an alcoholic extract, preferably a methanol extract. The extract is preferably produced by a process comprising the steps of
extraction of black currants and/or bilberries,
purification via chromatography,
mixing of the extract(s) with water and
spray-drying of the mixture.

One example of such a process is disclosed in EP1443948.

In a preferred embodiment, maltodextrin is added to the composition.

A further preferred embodiment is directed to a composition for use in reducing immune cell activation, wherein the composition comprises delphinidin 3-rutinoside (D3R) having the following formula:

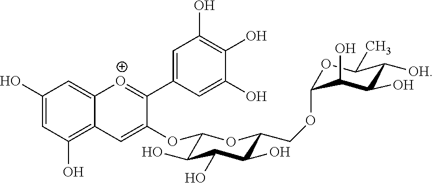

It is also intended to include pharmaceutically acceptable polymorphs, prodrugs, isomers, salts and derivatives of D3R.

In a further preferred embodiment of the invention the one or more anthocyanins in the anthocyanin composition is the D3R, and/or the extract of black currants and/or bilberries comprises the D3R.

D3R, also known as tulipanin is an anthocyanin found in black currants and other fruits and flowers can be used from a natural origin or can be synthesized in vitro or in vivo. D3R can be found in Peruvian lily (Alstroemeria spp.), berberis (Berberis spp.), princess vine (Cissus sicyoides), Hymenocallis (Hymenocallis spp.), Cassava (Manihot utilissima), Musa acuminata, dwarf lilyturf (Ophiopogon japonicus), Petunia exserta, Petunia reitzii, black currant (Ribes nigrum), Rye (Secale cereal), tamarillo (Solanum betaceum), Thaumatococcus danieffii, Tulipa spp. and in eggplants. It is preferred to use extracts of fruits, preferably of black currants as a source of D3R.

In an alternative embodiment, it is preferred to use cyanidin 3-rutinoside (C3R). C3R is also known as antirrhinin and can be found in various fruits, such as black currant (Ribes nigrum), açai (açai palm), black raspberry (Rubus leucodermis, Rubus occidentalis, Rubus coreanus), lychee pericarp (Litchi chinensis) and common fig (Ficus carica). It is preferred to use extracts of fruits, preferably of black currants or black raspberry as a source of C3R.

The composition according to the present invention preferably contains at least three monosaccharide anthocyanins. Moreover, it preferably contains at least one monosaccharide anthocyanin in which the saccharide is arabinose or at least one disaccharide anthocyanin in which the disaccharide is rutinose. The composition preferably contains anthocyanins with at least two different aglycones, more preferably at least four. Especially preferably the composition contains anthocyanins in which the aglycone units are cyanidin, peonidin, delphinidin, petunidin, malvidin and optionally also pelargonidin. In one preferred embodiment, the composition also contains at least one trisaccharide anthocyanin. The disaccharide anthocyanins are more water-soluble than the monosaccharides; moreover, cyanidin and delphinidin anthocyanins are amongst the most water-soluble anthocyanins.

In an advantageous embodiment of the present invention anthocyanins are selected from cyanidin-3-glucoside, cyanidin-3-galactoside, cyanidin-3-arabinoside, delphinidin-3-glucoside, delphinidin-3-galactoside, delphinidin-3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside. The anthocyanins are preferably selected from cyanidin-3-glucoside, cyanidin-3-rutinoside, delphinidin-3-glucoside, delphinidin-3-rutinoside, cyanidin-3-galactoside, delphinidin-3-galactoside.

It is especially preferred according to the present invention to use a composition containing one or more of the following: cyanidin-3-glucoside, delphinidin-3-glucoside, cyanidin-3-rutinoside, delphinidin-3-rutinoside, preferably containing all four anthocyanins.

The anthocyanins can be from natural sources or from synthetic productions. Natural sources are preferably selected from fruits, flowers, leaves, stems and roots, preferably violet petal, seed coat of black soybean. Preferably anthocyanins are extracted from fruits selected from: açai, black currant, aronia, eggplant, blood orange, marion blackberry, black raspberry, raspberry, wild blueberry, cherry, queen Garnet plum, red currant, purple corn (Z. mays L.), concord grape, norton grape, muscadine grape, red cabbage, okinawan sweet potato, Ube, black rice, red onion, black carrot. Particularly suitable fruit sources for the anthocyanins are cherries, bilberries, blueberries, black currants, red currants, grapes, cranberries, strawberries, black chokeberry, and apples and vegetables such as red cabbage. Bilberries, in particular Vaccinium myrtillus, and black currants, in particular Ribes nigrum, are especially suitable. It is further preferred to use plants enriched with one or more of anthocyanins as natural sources, preferably plants enriched with delphinidin-3-rutinoside.

The counterion in the anthocyanins in the composition of the invention may be any physiologically tolerable counter anions, e.g. chloride, succinate, fumarate, malate, maleate, citrate, ascorbate, aspartate, glutamate, etc. Preferably however the counterion is a fruit acid anion, in particular citrate, as this results in the products having a particularly pleasant taste. Besides the anthocyanins, the composition may desirably contain further beneficial or inactive ingredients, such as vitamins (preferably vitamin C), flavones, isoflavones, anticoagulants (e.g. maltodextrin, silica, etc.), desiccants, etc.

The composition according to the present invention comprises all metabolization that can occur in a PBMC cell physiological condition, having an oral bioavailability.

Moreover, it is preferred, when the subject is being treated or has been treated with an anticancer treatment. Specifically, the composition is to be administered simultaneously, separately or sequentially with an anticancer treatment. In other words, the anticancer treatment is to be administered alongside the treatment with the composition of the present invention.

In a preferred configuration, the anticancer treatment is a chemotherapy, radiotherapy, steroid therapy, targeted therapy, photodynamic therapy, biologic therapy, immunotherapy, CAR T therapy, or stem cell therapy. Therefore, it is preferred, when the composition further comprises a chemotherapeutic drug.

In a preferred embodiment of the present invention, the composition is for use in reducing immune cell activation. More preferably the reduction of immune cell activation is a reduction of unspecific immune cell activation, or a prevention of immune cell activation or a reduction of the release of proinflammatory cytokines.

The reduction of unspecific immune response is a non-receptor-based IL-2-mediated immune response, in contrast to a receptor cofactor mediated activation via UCHT1 (anti-CD3) antibody.

Most importantly, the reduction of immune cell activation is a reversible, non-immune suppressive, non-toxic reduction of activation and/or proliferation of mammalian Peripheral blood mononuclear cells (PBMC), preferably mammalian T cells.

A further aspect of the present invention is related to a composition for use according to the present invention, wherein in an in vitro assay with PBMCs from healthy humans suitable to determine the ability of the composition to reduce immune cell activation, the composition generates at least 30% less, preferably at least 40% less, more preferably at least 50% less cell proliferation than a negative control with water alone, wherein the in vitro assay comprises activating the PBMCs with anti-CD3 and anti-CD28, induction of proliferation by addition of IL-2 and incubating the PBMCs with the composition or the negative control, and determining the level of cell proliferation after a time period, which is at least 2 days, preferably 3 days.

A further aspect is the use according to the present invention in cell therapy and cell culture comprising any fraction of PBMC cells.

According to a further aspect of the present invention, the reduction of immune cell activation is a stabilization of non-activated state of the immune system, maintaining a healthy T cell phenotype and prevents an overshooting of immune cell population.

The disease or disorder is an autoimmune disease preferably selected from multiple sclerosis (MS), rheumatoid arthritis (RA), psoriasis, psoriatic arthritis, colitis ulcerosa, Crohn's disease, myasthenia gravis (MG), autoimmune polyglandular syndrome type II (APS-II), Hashimoto's thyroiditis (HT), type-1 diabetes (T1 D), systemic lupus erythematosus (SLE) and autoimmune lymphoproliferative syndrome (ALS), or an allergy, asthma, or an infectious disease. Further, there is evidence that Parkinson's disease is an autoimmune disorder, in which the immune system mistakenly attacks part of the body (Sulzer et al., Nature vol. 546, p. 656-661, 2017). Therefore, the present invention is also suitable for protection and/or treatment of Parkinson's disease. This shall also apply to bacterial opportunistic infections.

It is preferred when the composition comprises anthocyanins and is to be administered to the subject in a dose of the anthocyanins/regimen of 1 to 10 oral dosages of at least 80 mg anthocyanins each per day, preferably 3 to 6 oral dosages of at least 80 mg anthocyanins each per day.

In a further advantageous configuration, the composition is to be administered to the subject as parenteral bolus injection or infusion or parenteral nutritional solution. It is also preferred to use the composition to stabilize critical patients, where lifesaving treatments are not effective, and no last-line treatment is available (due to lack of treatment options).

The composition according to the present invention can be administered by oral or parenteral means or by organ injection, preferably in lymph nodes or thymus. A formulation to be administered can be a micro- or nano-formulation (e.g. liposomal formulation or polymer particles) targeting the cytokine network.

The composition according to the present invention is to be administered to the subject, reaching a concentration in the target compartment at least 30 μg/ml, preferably at least 100 μg/ml. Target compartment are blood and lymph, specifically the medium surrounding the cells of the immune system, preferably Peripheral Blood Mononuclear Cells (PBMCs).

The invention is further directed to a combined preparation for use in treating or preventing a cancer in a subject, wherein the cancer comprises cancer cells that are CD4+, wherein the combined preparation comprises a composition and an anticancer treatment, wherein the composition is as defined above and in particular as defined in any of claims 8 to 13, wherein the composition and the anticancer treatment are to be administered separately, simultaneously or sequentially.

Moreover, it is directed to a composition comprising: (i) one or more of an extract of black currants, an extract of bilberries, and an anthocyanin; and (ii) an oral chemotherapeutic drug, optionally wherein the oral chemotherapeutic drug is bexarotene, chlorambucil, cyclosporine, cyclophosphamide, doxorubicin, fludarabine, methotrexate, pentostatin, prednisone, vincristine.

The composition according to the present invention may be in tablet, capsule or liquid form. The composition may comprise one or more of an extract of black currants, an extract of bilberries, and an anthocyanin, preferably selected from cyanidin-3-glucoside, cyanidin-3-galactoside, cyanidin-3-arabinoside, delphinidin-3-glucoside, delphinidin-3-galactoside, delphinidin-3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside, more preferably selected from cyanidin-3-glucoside, delphinidin-3-glucoside, cyanidin-3-rutinoside and delphinidin-3-rutinoside

The invention is further directed to a combined preparation comprising an analgesic or an anti-inflammatory agent and an extract of black currants and/or bilberries or the anthocyanin composition defined herein, for simultaneous, separate or sequential use in medicine, preferably wherein the analgesic is ibuprofen or paracetamol/acetaminophen or a nonsteroidal anti-inflammatory drug (NSAID).

Analgesic compounds are preferably selected from acetylsalicylic acid, Diclofenac, Dexibuprofen, Dexketoprofen, Flurbiprofen, Ibuprofen, Indometacin, Ketoprofen, Meloxicam, Nabumeton, Naproxen, Phenylbutazon, Piroxicam, Phenazon, Propyphenazon, rofecoxib, Celecoxib, Etoricoxib, Parecoxib, Metamizol, Paracetamol/Acetaminophen.

The invention also refers to a combined preparation comprising an immune system suppressant and an extract of black currants and/or bilberries or the anthocyanin composition defined herein, for simultaneous, separate or sequential use in medicine, preferably wherein the immune system suppressant is methotrexate, a glucocorticoid selected from prednisone, dexamethasone, hydrocortisone or a TNF inhibitor.

### Item List

Preferred embodiments of the present invention are summarized in the following item list:

1. A composition for use in treating or preventing a cancer in a subject, wherein the cancer comprises cancer cells that are CD4+, and wherein the composition comprises one or more of an extract of black currants, an extract of bilberries, and an anthocyanin.

2. The composition for use according to item 1, wherein the cancer is a T-cell cancer.

3. The composition for use according to item 1 or 2, wherein the cancer is a T-cell leukemia or a T cell lymphoma.

4. The composition for use according to item 3, wherein the cancer is Sézary syndrome, Hodgkin's lymphoma, dermatopathic lymphadenitis, indolent MCL (mantle cell lymphoma).

5. The composition for use according to any preceding item, wherein a blood sample taken from the subject comprises more than 1200 CD4+ cells/μL, optionally more than 1600 CD4+ cells/μL.

6. The composition for use according to any preceding item, wherein a blood sample taken from the subject has a CD4+/CD8+ cell ratio above 2, optionally above 2.5.

7. The composition for use according to any preceding item, wherein the composition reduces the CD4+/CD8+ cell ratio.

8. The composition for use according any preceding item, wherein the black currants are the fruit of Ribes nigrum and/or the bilberries are the fruit of Vaccinium myrtillus.

9. The composition for use according to any preceding item wherein the composition contains an extract from black currants and bilberries in a weight ratio of 0.5:1 to 1:0.5.

10. The composition for use according to any preceding item wherein the composition is an extract of the pomaces from black currants and bilberries, preferably an alcoholic extract.

11. The composition for use according to any preceding item, wherein the composition comprises anthocyanins and the anthocyanins are present in the composition at a concentration of at least 25 weight-%.

12. The composition for use according to any preceding item, wherein the extract is prepared by a process comprising the steps of extraction of black currants and/or bilberries, purification via chromatography, mixing of the extract(s) with water and spray-drying of the mixture.

13. A composition for use according to item 8, further comprising one or more of the following:

cyanidin-3-glucoside, delphinidin-3-glucoside, cyanidin-3-rutinoside.

14. The composition for use according to any preceding item, comprising one or more of the following anthocyanins:
cyanidin-3-glucoside, cyanidin-3-galactoside, cyanidin-3-arabinoside, delphinidin-3-glucoside, delphinidin-3-galactoside, delphinidin -3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside, preferably comprising cyanidin-3-glucoside, delphinidin-3-glucoside, cyanidin-3-rutinoside and delphinidin-3-rutinoside.

15. The composition for use according to any preceding item, wherein the anthocyanin is delphinidin 3-rutinoside.

16. The composition for use according to any preceding item, wherein the subject is being treated or has been treated with an anticancer treatment.

17. The composition for use according to any preceding item, wherein the composition is to be administered simultaneously, separately or sequentially with an anticancer treatment.

18. The composition for use according to item 16 or 17, wherein the anticancer treatment is a chemotherapy, radiotherapy, steroid therapy, targeted therapy, photodynamic therapy, biologic therapy, immunotherapy CAR T therapy, or stem cell therapy.

19. The composition for use according to any preceding item, wherein the composition further comprises a chemotherapeutic drug.

20. A composition for use according to any preceding item, wherein in an in vitro assay with PBMCs from healthy humans suitable to determine the ability of the composition to reduce immune cell activation, the composition generates at least 30% less, preferably at least 40% less, more preferably at least 50% less cell proliferation than a negative control with water alone, wherein the in vitro assay comprises activating the PBMCs with anti-CD3 and anti-CD28, induction of proliferation by addition of IL-2 and contacting the PBMCs with the composition or the negative control, and determining the level of cell proliferation after a time period, which is preferably 3 days.

21. A composition for use according to any preceding item in cell therapy and cell culture comprising any fraction of PBMC cells.

22. A composition for use according to any preceding item, wherein the reduction of immune cell activation is a stabilization of non-activated state of the immune system.

23. A composition for use according to any preceding item, the disease or disorder is an autoimmune disease preferably selected from multiple sclerosis (MS), rheumatoid arthritis (RA), psoriasis, psoriatic arthritis, colitis ulcerosa, Crohn's disease, myasthenia gravis (MG), autoimmune polyglandular syndrome type II (APS-II), Hashimoto's thyroiditis (HT), type-1 diabetes (T1 D), systemic lupus erythematosus (SLE), autoimmune lymphoproliferative syndrome (ALS), Parkinson's disease, or an allergy, asthma, or an infectious disease.

24. The composition for use according to any preceding item wherein the composition comprises anthocyanins and is to be administered to the subject 1 to 10 oral dosages of at least 80 mg anthocyanins each per day, preferably 3 to 6 oral dosages of at least 80 mg anthocyanins each per day.

25. The composition for use according to any preceding item wherein the composition is to be administered to the subject as parenteral bolus injection or infusion or parenteral nutritional solution to stabilize critical patients.

26. The composition for use according to any preceding item wherein the composition is to be administered by oral or parenteral means or by organ injection, preferably in lymph nodes or thymus.

27. The composition for use according to any preceding item wherein the composition is to be administered to the subject, reaching a concentration in the target compartment of at least 30 μg/ml, preferably at least 100 μg/ml.

28. A combined preparation for use in treating or preventing a cancer in a subject, wherein the cancer comprises cancer cells that are CD4+, wherein the combined preparation comprises a composition and an anticancer treatment, wherein the composition is as defined in any of claims 1 to 16, wherein the composition and the anticancer treatment are to be administered separately, simultaneously or sequentially.

29. A combined preparation comprising (i) one or more of an extract of black currants, an extract of bilberries, and an anthocyanin; and (ii) an oral chemotherapeutic drug, optionally wherein the oral chemotherapeutic drug is bexarotene, chlorambucil, cyclosporine, cyclophosphamide, doxorubicin, fludarabine, methotrexate, pentostatin, prednisone, vincristine.

30. The composition according to item 29, wherein the composition is in tablet, capsule or liquid form.

31. The composition according to claim 21 or claim 22, wherein the one or more of an extract of black currants, an extract of bilberries, and an anthocyanin are as defined in any of claims 8 to 13.

32. A method for treating or preventing a cancer in a subject, comprising administering a composition to the subject, wherein the cancer comprises cancer cells that are CD4+, and wherein the composition comprises one or more of an extract of black currants, an extract of bilberries, and an anthocyanin.

33. A method for reducing immune cell activation in a subject, which comprises administering to a subject in need thereof a composition comprising one or more of an extract of black currants, an extract of bilberries, and an anthocyanin.

34. A method for reducing the number of CD4+ cells in a subject, which comprises administering to a subject in need thereof a composition comprising one or more of an extract of black currants, an extract of bilberries, and an anthocyanin, so as to reduce the number of CD4+ cells, optionally wherein the subject has a cancer comprising cancer cells that are CD4+ cells, further optionally wherein in a blood sample taken from a subject comprises more than 1200 cells/μL CD4+ T cells.

35. A method according to any of items 32 to 34, wherein the composition is as defined in any of items 8 to 13.

36. A method according to any of items 32 to 35, wherein the subject is suffering from cancer and the subject or the cancer are as defined in any of items 1 to 7.

### EXAMPLES

The berry extract composition (Healthberry® 865; Evonik Nutrition & Care GmbH, Darmstadt, Germany) used in the present study is a dietary supplement consisting of 17 purified anthocyanins (all glycosides of cyanidin, peonidin, delphinidin, petunidin, and malvidin) isolated from black currant (Ribes nigrum) and bilberries (Vaccinium myrtillus).

The relative content of each anthocyanin in the Healthberry® 865 product was as follows: 33.0% of 3-O-b-rutinoside, 3-O-b-glucosides, 3-O-b-galactosides, and 3-O-b-arabinosides of cyanidin; 58.0% of 3-O-b-rutinoside, 3-O-b-glucosides, 3-O-b-galactosides, and 3-O-b-arabinosides of delphinidin; 2.5% of 3-O-b-glucosides, 3-O-b-galactosides, and 3-O-b-arabinosides of petunidin; 2.5% of 3-O-b-glucosides, 3-O-b-galactosides, and 3-O-b-arabinosides of peonidin; 3.0% of 3-O-b-glucosides, 3-O-b-galactosides, and 3-O-b-arabinosides of malvidin.

The 3-O-b-glucosides of cyanidin and delphinidin constituted at least 40-50% of the total anthocyanins.

The major anthocyanins contained in the berry extract used are cyanidin-3-glucoside, cyanidin-3-rutinoside, delphinidin-3-glucoside, delphinidin-3-rutinoside, cyanidin-3-galactoside and delphinidin-3-galactoside.

In addition to the anthocyanins mentioned above, the product also contained maltodextrin (around 40 weight-% of the composition), and citric acid (to maintain stability of anthocyanins). The amount of anthocyanin citrate is at least 25 weight-% of the composition. The composition is prepared from black currants and bilberries by a process comprising the steps of alcoholic extraction of black currants and bilberries, purification via chromatography, mixing of the extracts with maltodextrin citrate and water and spray-drying of the mixture. The product composition contains extracts of black currants and bilberries mixed in a weight ratio of around 1:1.

### Materials

TABLE 1
Materials used for the measurement
of cell survival and metabolism

| Material | Supplier |
|---|---|
| RealTime-Glo ™ | Promega GmbH, Mannheim (Germany) |
| MT Cell Viability | |
| Assay | |
| CellTiter-Glo | Promega GmbH, Madison (USA) |
| Luminescent Cell | |
| Viability Assay, | |
| (Cat. No G7570) | |
| Dulbecco's | Gibco Life technologies, Carlsbad (USA) |
| Modified Eagle's | |
| medium (DMEM) | |
| Fetal bovine serum | Gibco Life technologies, Carlsbad (USA) |
| Vero SLAM cells | ATCC/American Type Culture Collection in |
| | Partnership with LGC standards, Wesel (Germany) |
| LL-MK2 cells | ATCC/American Type Culture Collection in |
| | Partnership with LGC standards, Wesel (Germany) |
| HeLa cells | ATCC/American Type Culture Collection in |
| | Partnership with LGC standards, Wesel (Germany) |
| THP 1 cells | ATCC/American Type Culture Collection in |
| | Partnership with LGC standards, Wesel (Germany) |
| Jurkat cells | ATCC/American Type Culture Collection in |
| | Partnership with LGC standards, Wesel (Germany) |
| Mt4 cells | ATCC/American Type Culture Collection in |
| | Partnership with LGC standards, Wesel (Germany) |
| Healthberry ® 865 | Evonik Nutrition & Care GmbH, Darmstadt |
| (anthocyanin | (Germany) |
| content 29.7%) | |

TABLE 2
Devices used for the measurement of cell survival and metabolism.

| Device | Supplier |
|---|---|
| Centro LB 960 microplate | Berthold Technologies, (Germany) |
| luminometer | |

TABLE 3
Materials used for cytokine release assay

| Material | Supplier |
|---|---|
| THP-1 cells LOT 30 ATCC | DSMZ, Braunschweig (Germany) |
| Lipopolysaccharide (LPS) | Invitrogen, Carlsbad (USA) |
| solution (500×) LOT | |
| 4351181 | |
| Roswell Park Memorial | Gibco Life technologies, Carlsbad (USA) |
| Institute medium (RPMI | |
| 1640) | |
| Bovine serum albumin/ | Carl Roth, Karlsruhe (Germany) |
| Albumin Fraktion V | |
| (≥98%, pulv., for | |
| molecular biology) 200 g | |
| Fetal bovine serum | Gibco Life technologies, Carlsbad (USA) |
| Sodium Pyruvate MEM 100 | Gibco Life technologies, Carlsbad (USA) |
| mM 100 mL | |
| Gentamicin (50 mg/mL) 10 | Thermo Fisher Scientific GmbH, Dreieich |
| mL | (Germany) |
| Human IL-6 ELISA kit | Sigma Aldrich, St. Louis (USA) |
| (Cat. No RAB0306-1KT) | |
| Human IL-1 β ELISA kit | Sigma Aldrich, St. Louis (USA) |
| (Cat. No RAB0273-1KT) | |
| Human TNF-α ELISA kit | Sigma Aldrich, St. Louis (USA) |
| (Cat. No RAB0476-1KT) | |
| Healthberry ® 865 | Evonik Nutrition & Care GmbH, |
| (anthocyanin | Darmstadt (Germany) |
| content 29.7%) | |

TABLE 4
Devices used for cytokine release assay.

| Device | Supplier |
|---|---|
| Safety cabinet HERA Safe | Thermo Fisher Scientific GmbH, Dreieich |
| 2020 | (Germany) |
| Automatic cell counter | Thermo Fisher Scientific GmbH, Dreieich |
| Countess | (Germany) |
| Centrifuge 5415R | Eppendorf, Hamburg (Germany) |
| Attune Nxt | Thermo Fisher Scientific GmbH, Dreieich |
| | (Germany) |
| Heating thermo shaker | DITABIS Digital Biomedical Imaging |
| HTMR 133 | Systems AG, Pforzheim (Germany) |
| Vortex- Genie 2 | Scientific Industries, Bohemia (USA) |
| TECAN Infinite 200 Pro | Tecan Group Ltd., Mannedorf (Switzerland) |

TABLE 5
Materials used for T-cell activation assays
and analysis of the T-cell population

| Material | Supplier |
|---|---|
| PBMCs (peripheral blood | Derived from human blood |
| monocytic cells) | |
| Cell trace ™ CFSE | Invitrogen, Carlsbad (USA) |
| Annexin V_Alexa594 | Invitrogen, Carlsbad (USA) |
| Roswell Park Memorial | Gibco Life technologies, Carlsbad (USA) |
| Institute medium (RPMI | |
| 1640) | |
| Fetal bovine serum | Gibco Life technologies, Carlsbad (USA) |
| HEPES stock solution (1M) | Gibco Life technologies, Carlsbad (USA) |
| Penicillin-Streptomycin | Gibco Life technologies, Carlsbad (USA) |
| β-Mercaptoethanol | Sigma Aldrich, St. Louis (USA) |
| Interleukin 2 | Peprotech GmbH, Hamburg (Germany) |
| UCHT1 (anti-CD3) antibody | BioLegend, Koblenz (Germany) |
| Human anti-CD-28.2 | BioLegend, Koblenz (Germany) |
| Anti-CD4_APC | BioLegend, Koblenz (Germany) |
| Anti-CD8_PE | BioLegend, Koblenz (Germany) |
| Healthberry ® 865 | Evonik Nutrition & Care GmbH |
| (anthocyanin | |
| content 29.7%) | |
| Bilberry extract, Vaccinium | Evonik Nutrition & Care GmbH, |
| myrtillus (anthocyanin | Darmstadt (Germany) |
| content 38.8%) | |
| Black currant extract, Ribes | Evonik Nutrition & Care GmbH, |
| nigrum (anthocyanin content | Darmstadt (Germany) |
| 30%) | |
| Berry extract analogue to | Evonik Nutrition & Care GmbH, |
| Healthberry ® 865 without | Darmstadt (Germany) |
| maltodextrin | |
| GLUCIDEX IT 19 | ROQUETTE GmbH, Frankfurt (Germany) |
| (maltodextrin) | |
| Delphinidin 3-rutinoside | Polyphenols AS, Sandnes (Norway) |
| Delphinidin 3-glucoside | Polyphenols AS, Sandnes (Norway) |
| Cyanidin 3-rutinoside | Polyphenols AS, Sandnes (Norway) |
| Cyanidin 3-glucoside | Polyphenols AS, Sandnes (Norway) |
| Astaxanthin | Sigma-Aldrich Chemie GmbH, (Munich, |
| | Germany) |
| Rosemary Antioxidans | Flavex Naturextrakte GmbH, |
| Extrakt 25 | Saarbrücken (Germany) |

TABLE 6
Devices used for the T-cell activation assays
and analysis of the T-cell population.

| Device | Supplier |
|---|---|
| Attune Nxt | Thermo Fisher Scientific GmbH, Dreieich |
| | (Germany) |
| Heraeus Multifuge X1R | Thermo Fisher Scientific GmbH, Dreieich |
| | (Germany) |
| Heracell 240i incubator | Thermo Fisher Scientific GmbH, Dreieich |
| | (Germany) |

### Methods

### Measurement of Cell Survival and Metabolism

Cell survival and metabolism was measured by RealTime-Glo™ MT Cell Viability Assay. Vero-Slam and LL-MK2 cells (2×10⁴) were incubated with decreasing amounts of the test compound solubilized in DMEM. Wells with DMEM alone served as control. The MT Cell Viability Substrate and the NanoLuc luciferase were added according to the manufacturer's instructions. The assays were performed in triplicates according to the manufacturer's instructions. After 1 h, and then every six or 12 h, the luminescence was measured with Centro LB 960 microplate luminometer (Berthold Technologies, Germany). Luminescence values after 1 h were set to 1 and changes over time were calculated.

Similar cell viability assays were performed with additional cell lines (HeLa, Jurkat, Mt4 and THP-1) and using the Cell Titer Glo Assay.

### Cytokine Release Assay

The assay used THP-1 cells, a monocyte suspension cell line, as starting point. In a first step, the monocytes were differentiated into macrophages via incubation with 50 nM PMA (phorbol 12-myristate 13-acetate) for 24 h followed by a resting period of 24 h in PMA free media in a CO₂-Incubator (37° C., 5% CO₂, 95% humidity). Resulting macrophages adhere to the surface of cell culture plastic ware and could be used for the cytokine release assay.

On the first day of the assay, 7.5×10⁵ cells per well were seeded in a 6-well plate and differentiated into macrophages as described. During the resting time of 24 hours without PMA, Healthberry® 865 was added in a concentration of 25 μg/mL to investigate its influence on cytokine release and immune reaction. This scenario should simulate the preventative consumption of the product (addition of Healthberry® 865 prior to the LPS stimulus). The control was rested in medium without Healthberry® 865. After the resting period, immune reaction was triggered by addition of 100 ng/mL LPS. After 24 hours of LPS exposure, cell culture medium was removed and prepared for cytokine measurement by ELISA.

1 mL of cell culture medium was used and centrifuged for 10 min at 1000×g. Cell culture supernatants were stored at −20° C. until further usage for cytokine measurement. Single cytokine ELISAs for IL-6, IL-1β and TNF-α were used for the quantification of the respective cytokines in the cell culture supernatant. Absorbance was measure with a multiplate reader at 450 nm. Resulting cytokine amount was normalized to the number of cells in the sample after the assay procedure.

### T Cell Activation Assay

Resting, unstimulated T cells are in the G₀ phase of the cell cycle and do not proliferate. Upon recognition of a foreign substance (foreign peptide presented on MHC) by the T cell receptor (TCR) and stimulation of the co-receptor CD28 the T cells start to proliferate. Thus, proliferation is a clear indication of T cell activation. Another classic activation marker for T cells is CD25, the high affinity receptor for the interleukin IL-2. This protein is not expressed by resting T cells and transcription is induced by TCR signaling. Thus, the presence of CD25 on the surface of T cells is a clear indicator of T cell activation. On the contrary, T cell exhaustion correlates with the signaling-induced expression of PD-1 on the surface of the T cells. To test the impact of substances on T cell activation, proliferation as well as CD25 and PD-1 upregulation, are used as readouts. Primary human Peripheral blood mononuclear cells (PBMCs) isolated from the blood of healthy donors (including primary T-cells) were used. PBMC cells represent a heterogenic population with 45-80% T cells, 5-20% B cells, 10-25% monocytes and 5-20% NK cells.

### CellTrace™ CFSE Cell Proliferation Staining

CellTrace™ stock solution was prepared according to the manual; addition of 15 μL DMSO to the CellTrace™ reagent and mixed well. Cell suspension was adjusted to 10⁶ cells/mL in RPMI without FBS before addition of 1 μL CellTrace™ stock solution to each mL of cell suspension. After 20 minutes incubation (protected from light) of the cells at room temperature, five times the volume of culture medium was added and then again incubated for 5 min at room temperature. Afterwards the cells were centrifuged (300×g for 5 min) and the pellet resuspended in fresh culture medium. As last step the cells were incubated at 37° C. with 5% CO₂ for 1-2 hours before the set-up of the experiment.

### Preparation of Test Samples

Samples of the berry extracts were prepared in water, Astaxanthin in DMF and the anthocyanins C3R, D3R, C3G and D3G as well as Rosemary extract in DMSO.

TABLE 7
Overview test samples and dilutions.

| Test Substance | 10X conc. [μg/ml] Dilution series in H₂O | | | | | | | | | | |
|---|---|---|---|---|---|---|---|---|---|---|---|
| Healthberry with | 5000 | 2500 | 1000 | 500 | 250 | 100 | 50 | 25 | 12.5 | 5 | 2.5 |
| Maltodextrin | | | | | | | | | | | |
| Berry extract | 3000 | 1500 | 600 | 300 | 150 | 60 | 30 | 15 | 7.5 | 3 | 1.5 |
| analogue without | | | | | | | | | | | |
| Maltodextrin | | | | | | | | | | | |
| Maltodextrin | 5000 | 1000 | 400 | 200 | 100 | 40 | 20 | 10 | 5 | 2 | 1 |
| Black current extract | 5000 | 1665 | 666 | 333 | 166.5 | 66.6 | 33.3 | 16.65 | 8.325 | 3.33 | 1.665 |
| Bilberry extract | 5000 | 1665 | 666 | 333 | 166.5 | 66.6 | 33.3 | 16.65 | 8.325 | 3.33 | 1.665 |
| Astaxanthin | 500 | 100 | 40 | 20 | 10 | 4 | 2 | 1 | 0.5 | 0.2 | 0.1 |
| Cyanidin-3-O- | 1000 | 750 | 300 | 150 | 75 | 30 | 15 | 7.5 | 3.75 | 1.5 | 0.75 |
| rutinosid/C3R | | | | | | | | | | | |
| Delphinidin-3-O- | 1000 | 750 | 300 | 150 | 75 | 30 | 15 | 7.5 | 3.75 | 1.5 | 0.75 |
| rutinosid/D3R | | | | | | | | | | | |
| Cyanidin-3-O- | 333.3 | 250 | 100 | 50 | 25 | 10 | 5 | 2.5 | 1.25 | 0.5 | 0.25 |
| glucosid/C3G | | | | | | | | | | | |
| Delphinidin-3-O- | 333.3 | 250 | 100 | 50 | 25 | 10 | 5 | 2.5 | 1.25 | 0.5 | 0.25 |
| glucosid/D3G | | | | | | | | | | | |
| Delphinidin-3-O- | 333.3 | 250 | 100 | 50 | 25 | 10 | 5 | 2.5 | 1.25 | 0.5 | 0.25 |
| rutinosid/D3R | | | | | | | | | | | |
| Rosemary extract | 5000 | 2000 | 800 | 400 | 200 | 80 | 40 | 20 | 10 | 4 | 2 |

Following CFSE labelling and recovering at 37° C. with 5% CO₂, cells were mixed with the extract as follows: 20 μL of extract were placed in a 96-well u-bottom culture plate as triplicates, then 180 μL cell suspension was added on top and mixed. Final concentration of the cells was 5*10⁴ cells/well (=2.5*10⁵ cells/mL).

Rosemary extract was excluded for further experiments and evaluations due to too high in vitro toxicity.

### Flow Cytometry Measurements

Sample format: 96 well with 200 μL/well and 10,000 events measured per sample. FACS (fluorescence activated cell sorting) gating was performed via SSC (sideward scatter) versus FSC (forward scatter) density blot. A gate has been applied to identify viable cells. Percentage of viable cells was determined for all samples of a 96-well plate.

CellTrace™ CFSE median fluorescence (488 nm excitation and a 530/30 nm bandpass emission filter): From viable cell population single cells were determined and viable, single cells within the gate defined in a histogram to evaluate the level of fluorescence intensity of cells labelled with CellTrace™ CFSE. Cell proliferation was followed for 7 days. With increasing level of proliferation median fluorescence decreases, shown as shift of peak from right to left within the flow cytometer; non-labeled cells shown on the far-left side. Median fluorescence was determined as read-out. Earliest observable influence on proliferation and activation was expected after 3 days of incubation with the test compounds.

### Experiment to Test if the Samples Have an Activating Effect on Isolated PBMCs

All cells (40*10⁶) of one vial of frozen PBMC's (non-activated) were used for CFSE staining and cultured with extracts as follows: Day 0-3 in RPMI 1640 with 10% FBS, 1% HEPS (10 mM); day 3-7 in RPMI 1640 with 10% FBS, 1% HEPS (10 mM) and 100 U/mL IL-2.

Water, DMSO or DMF were used as negative control; UCHT1 (anti-CD3) antibody as positive control. The samples plate set-up was used as follows:

TABLE 8
Overview plate set-up test samples and dilutions for testing the activating effect.

| | | Final conc. [μg/ml] | | | | | | | | | | | | |
|---|---|---|---|---|---|---|---|---|---|---|---|---|---|---|
| Test Substance | | 1 | 2 | 3 | 4 | 5 | 6 | 7 | 8 | 9 | 10 | 11 | 12 | |
| Healthberry with | A | 250 | 250 | 250 | 100 | 100 | 100 | 50 | 50 | 50 | 1.5 | 1.5 | 1.5 | DMSO [%] |
| Maltodextrin | | | | | | | | | | | | | | |
| Berry extract | B | 150 | 150 | 150 | 60 | 60 | 60 | 30 | 30 | 30 | 0.6 | 0.6 | 0.6 | DMSO [%] |
| analogue without | | | | | | | | | | | | | | |
| Maltodextrin | | | | | | | | | | | | | | |
| Maltodextrin | C | 100 | 100 | 100 | 40 | 40 | 40 | 20 | 20 | 20 | 0.3 | 0.3 | 0.3 | DMSO [%] |
| Black current extract | D | 167 | 167 | 167 | 66.6 | 66.6 | 66.6 | 33.3 | 33.3 | 33.3 | | | | |
| Bilberry extract | E | 167 | 167 | 167 | 66.6 | 66.6 | 66.6 | 33.3 | 33.3 | 33.3 | | | | |
| Cyanidin-3-O- | F | 75 | 75 | 75 | 30 | 30 | 30 | 15 | 15 | 15 | | | | |
| rutinosid/C3R | | | | | | | | | | | | | | |
| Delphinidin-3-O- | G | 75 | 75 | 75 | 30 | 30 | 30 | 15 | 15 | 15 | | | | |
| rutinosid/D3R | | | | | | | | | | | | | | |
| C3G + D3G + D3R | H | 75 | 75 | 75 | 30 | 30 | 30 | 15 | 15 | 15 | | | | |
| Astaxanthin | A | 10 | 10 | 10 | 4 | 4 | 4 | 2 | 2 | 2 | | | | |
| DMF [% ] | B | 1.5 | 1.5 | 1.5 | 0.6 | 0.6 | 0.6 | 0.3 | 0.3 | 0.3 | | | | |
| UCHT1 | C | 5 | 5 | 5 | 2 | 2 | 2 | 1 | 1 | 1 | | | | |
| H₂O | D | | | | | | | | | | | | | |

Half medium change with fresh extracts of final concentration was done every day.

### Experiment to Test if the Samples Have an Influence on an Existing Activation of PBMCs and Therefore on Proliferation

PBMCs isolated from blood were activated for 2 days on an activating surface with anti-CD3 (UCHT1) and anti-CD28. Activated PBMCs were then induced to proliferate by adding 1000 U/ml IL-2. Therefore, the cells were transferred to a new dish without activating antibodies and 2 days later the activated cells were used to set up the experiment. 40*10⁶ cells were used for CFSE staining and cultured with extracts in RPMI 1640 with 10% FBS, 1% HEPS (10 mM), 0.1% β-mecaptoethanol (50 μM), 0.5% Pen/Strep and 100 U/ml IL-2.

Water, DMSO or DMF were used as negative control; UCHT1 (anti-CD3) antibody as positive control. The samples plate set-up was used as follows:

TABLE 9
Overview plate set-up test samples and dilutions for testing an influence on activation.

| | | Final conc. [μg/ml] | | | | | | | | | | | | |
|---|---|---|---|---|---|---|---|---|---|---|---|---|---|---|
| Test Substance | | 1 | 2 | 3 | 4 | 5 | 6 | 7 | 8 | 9 | 10 | 11 | 12 | |
| Healthberry with | A | 250 | 250 | 250 | 100 | 100 | 100 | 50 | 50 | 50 | 1.5 | 1.5 | 1.5 | DMSO [%] |
| Maltodextrin | | | | | | | | | | | | | | |
| Berry extract | B | 150 | 150 | 150 | 60 | 60 | 60 | 30 | 30 | 30 | 0.6 | 0.6 | 0.6 | DMSO [%] |
| analogue without | | | | | | | | | | | | | | |
| Maltodextrin | | | | | | | | | | | | | | |
| Maltodextrin | C | 100 | 100 | 100 | 40 | 40 | 40 | 20 | 20 | 20 | 0.3 | 0.3 | 0.3 | DMSO [%] |
| Black current extract | D | 167 | 167 | 167 | 66.6 | 66.6 | 66.6 | 33.3 | 33.3 | 33.3 | | | | |
| Bilberry extract | E | 167 | 167 | 167 | 66.6 | 66.6 | 66.6 | 33.3 | 33.3 | 33.3 | | | | |
| Cyanidin-3-O- | F | 75 | 75 | 75 | 30 | 30 | 30 | 15 | 15 | 15 | | | | |
| rutinosid/C3R | | | | | | | | | | | | | | |
| Delphinidin-3-O- | G | 75 | 75 | 75 | 30 | 30 | 30 | 15 | 15 | 15 | | | | |
| rutinosid/D3R | | | | | | | | | | | | | | |
| C3G + D3G + D3R | H | 75 | 75 | 75 | 30 | 30 | 30 | 15 | 15 | 15 | | | | |
| Astaxanthin | A | 10 | 10 | 10 | 4 | 4 | 4 | 2 | 2 | 2 | | | | |
| DMF [%] | B | 1.5 | 1.5 | 1.5 | 0.6 | 0.6 | 0.6 | 0.3 | 0.3 | 0.3 | | | | |
| UCHT1 | C | 5 | 5 | 5 | 2 | 2 | 2 | 1 | 1 | 1 | | | | |
| H₂O | D | | | | | | | | | | | | | |

Half medium change with fresh extracts of final concentration was done every day.

### Analysis of T Cell Populations Via Flow Cytometry (CD4+ & CD8+)

Sample preparation and plate set-up was used as described before in the T cell activation assay. Primary PBMCs were seeded on activating surface (anti-CD3, anti-CD28) with medium containing 1000 U/ml IL-2 for 2 days. The activated PBMCs were then cultured with the prepared sample concentrations in medium containing 100 U/ml IL-2 every day from day 3 on after isolation. Half medium change with fresh samples and flow cytometry measurement was performed every day. All cells for flow cytometry were labelled with an antibody mixture containing Annexin V_Alexa594, anti-CD4_APC and anti-CD8_PE for CD-marker as well as live-dead staining.

### Example 1: Influence of Berry Extracts on the Cellular Metabolism

To exclude cellular toxicity and adverse side effects, cellular viabilities of the test compounds on Vero-Slam and LL-MK2 cells (96 well-plate: 650 cells/well) were determined with the RealTime-Glo™ MT Cell Viability Assay kit. This assay measures the intracellular ATP content and therefore provides information on the cellular viability and metabolism. The cells were incubated with decreasing test compound concentration in triplicate assays. Subsequently, both the MT Cell Viability Substrate and NanoLuc® Enzyme were added, and the luciferase activities were measured after 1 h. These measurements were repeated every 6 h or 12 h, and changes to the luciferase activity at the start of the experiment were calculated per individual well. The luminescence was normalized on the mean of the medium control wells for each time-point. These compensations result in values of 1 for the medium control at each time point. Values less than 1 indicate a lower number of cells or a decrease in metabolic activity compared to the appropriate controls.

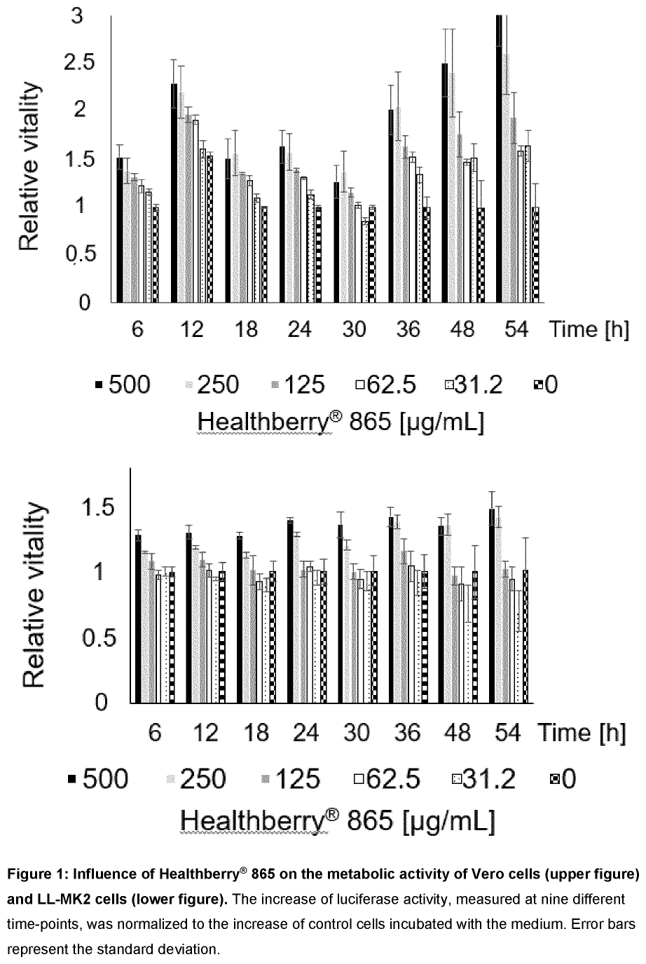

FIG. 1 displays the influence of Healthberry® 865 on the metabolic activity of Vero cells (upper figure) and LL-MK2 cells (lower figure). The increase of luciferase activity, measured at nine different time-points, was normalized to the increase of control cells incubated with the medium. Error bars represent the standard deviation.

Healthberry® 865 did not negatively influence cellular growth or metabolic activity at any concentration analysed, indicating the test compounds were non-toxic at these concentrations.

### Example 2: Effects of Berry Extracts on the Secretion of Cytokines

First, macrophages were obtained out of THP-1 monocytes via differentiation. When investigating the preventative scenario, Healthberry® 865 was added 24 hours before inducing immune reaction through LPS. Treatment with Healthberry® 865 stopped when LPS treatment was started (Healthberry® 865 treatment prior to LPS stimulus). To evaluate the level of immune reaction, the pro-inflammatory cytokines TNF-α, IL-1β and IL-6 were measured in cell culture supernatants.

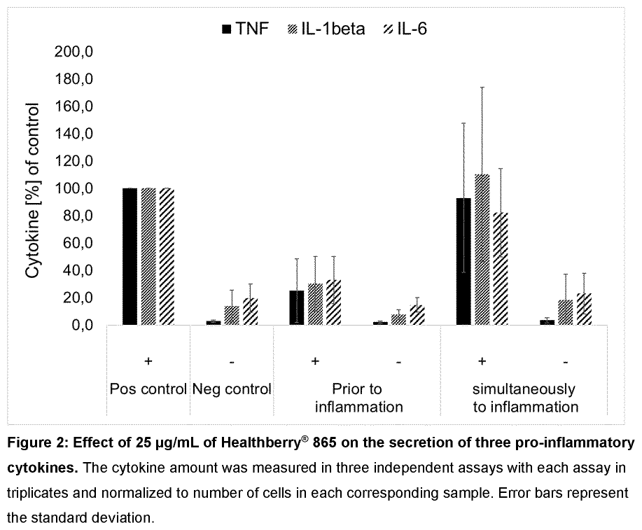

FIG. 2 shows the effect of 25 μg/mL of Healthberry® 865 on the secretion of three pro-inflammatory cytokines. The cytokine amount was measured in three independent assays with each assay in triplicates and normalized to number of cells in each corresponding sample. Error bars represent the standard deviation.

Positive and negative control were not exposed to Healthberry® 865 at any time and display the cytokine release triggered by lipopolysaccharides versus the stimulated baseline. Cells that were treated with Healthberry® 865 and then with LPS as an inflammatory stimulus are marked as “(+) prior to inflammation” and cells that were not exposed to LPS but likewise with the Healthberry® 865 “(−) prior to inflammation”. The latter served as a control to check if Healthberry® 865 itself caused cytokine release or interfered with the assay in another manner. This was excluded since cells that were treated with natural compounds showed the same cytokine profile as the negative control. In case of pre-treatment with Healthberry® 865 before the LPS stimulus, a significant reduction of the cytokine release could be observed. This confirms a significant effect of the berry extract of black currants and bilberries on the immune reaction.

### Example 3: Influence of Berry Extracts and Anthocyanins on the Activation of PBMCs

To evaluate, if the chosen test compounds could have an activating effect on isolated PBMCs (Peripheral Blood Mononuclear Cells) and induce them to proliferate, PBMCs were incubated together with the test compounds in different concentrations. The first 3 days no additional activation trigger was added to the cells, followed by the addition of Interleukin-2 for the following days (till day 7) as unspecific activation stimulus.

Based on cell viability determination the highest chosen test concentrations were defined as too high for the PBMCs (primary cells) used in this experiment, which was expected as primary cells in general are usually more sensitive than the cancer cell lines used in the previous examples. Without any activation, PBMCs usually display a high mortality rate (80-90%), which was also the case in the presence of the chosen test samples within the experiment.

Besides the evaluation of the cell viability, the activation of the cells was determined based on cell proliferation as read-out, which was measured via median fluorescence of CellTrace™ CFSE.

TABLE 10
Healthberry ® 865 and the other test samples themselves have no
activating effect on isolated PBMCs. Activation evaluated based on proliferation
and median CellTrace ™ CFSE as read-out (the lower the fluorescence
the higher the proliferation). Highest test concentrations should not be
considered due to lower viabilities. Average based on assay triplicates.

| | concentration | | | | |
|---|---|---|---|---|---|
| | [μg/mL]; | Median Fluorescence Day 2 | | Median Fluorescence Day 7 | |
| | DMSO & | | standard | | standard |
| | DMF [%] | Average | deviation | Average | deviation |
| Healthberry ® | 250 | 37563 | 1903 | 23917 | 1967 |
| 865 | 100 | 37235 | 197 | 22443 | 618 |
| | 50 | 35540 | 339 | 22775 | 137 |
| Berry extract | 150 | 34027 | 2016 | 28654 | 788 |
| analogue | 60 | 36363 | 102 | 21990 | 458 |
| | 30 | 35085 | 418 | 22246 | 436 |
| Black currant | 166.5 | 35446 | 2091 | 29441 | 4414 |
| extract | 66.6 | 35176 | 125 | 20801 | 290 |
| | 33.3 | 34059 | 461 | 21857 | 58 |
| Bilberry extract | 166.5 | 33942 | 7455 | 23223 | 4458 |
| | 66.6 | 35606 | 124 | 20457 | 233 |
| | 33.3 | 34239 | 428 | 21318 | 189 |
| Maltodextrin | 100 | 34548 | 146 | 22282 | 317 |
| | 40 | 34613 | 203 | 22196 | 50 |
| | 20 | 34299 | 222 | 22071 | 175 |
| UCHT1 positive | 5 | 35622 | 169 | 5899 | 4564 |
| control | 2 | 35172 | 159 | 6356 | 3255 |
| | 1 | 35226 | 445 | 8371 | 5323 |
| Water negative control | | 35761 | 193 | 21015 | 249 |
| C3R | 75 | 34486 | 174 | 22855 | 3592 |
| | 30 | 33737 | 258 | 21539 | 203 |
| | 15 | 33603 | 385 | 21030 | 204 |
| D3R | 75 | 37453 | 4311 | 44248 | n.a. |
| | 30 | 34646 | 354 | 25171 | n.a. |
| | 15 | 34772 | 44 | 20821 | 387 |
| Mixture C3G + | 75 | 33346 | 861 | n.a. | n.a. |
| D3G + D3R | 30 | 35435 | 371 | 21023 | 212 |
| | 15 | 34168 | 450 | 21014 | 587 |
| DMSO | 1.5 | 35561 | 261 | 21679 | 104 |
| | 0.6 | 35035 | 350 | 21754 | 450 |
| | 0.3 | 34344 | 318 | 21256 | 595 |
| Astaxanthin | 10 | 36178 | 2415 | 16442 | 1371 |
| | 4 | 32336 | 179 | 19444 | 201 |
| | 2 | 33336 | 179 | 20201 | 313 |
| DMF | 1.5 | 37515 | 1183 | 19272 | 538 |
| | 0.6 | 36628 | 377 | 21690 | 16 |
| | 0.3 | 36421 | 263 | 21916 | 71 |

| | | | | |
|---|---|---|---|---|
| Water negative control | 35761 | 193 | 21015 | 249 |

The results display no significant difference in proliferation after 2 days as well as till the maximum incubation time of 7 days in presence of the test compounds. Therefore, no immune cell activation per se, without any alternative activation stimulus, could be observed. For D3R even a slight decrease of proliferation and therefore activation could be observed. Only for the UCHT1 (anti-CD3) antibody used as positive control enhanced proliferation could be confirmed on day 7. These results confirm that all the tested samples including Healthberry® 865 do not activate PBMCs per se. These results indicate that no side effects of berry extracts of black currants and bilberries or anthocyanins on PBMC and T cell activation are expected without any other activating stimulus of the immune system (e.g. antigen presentation against a virus), which is an important safety aspect.

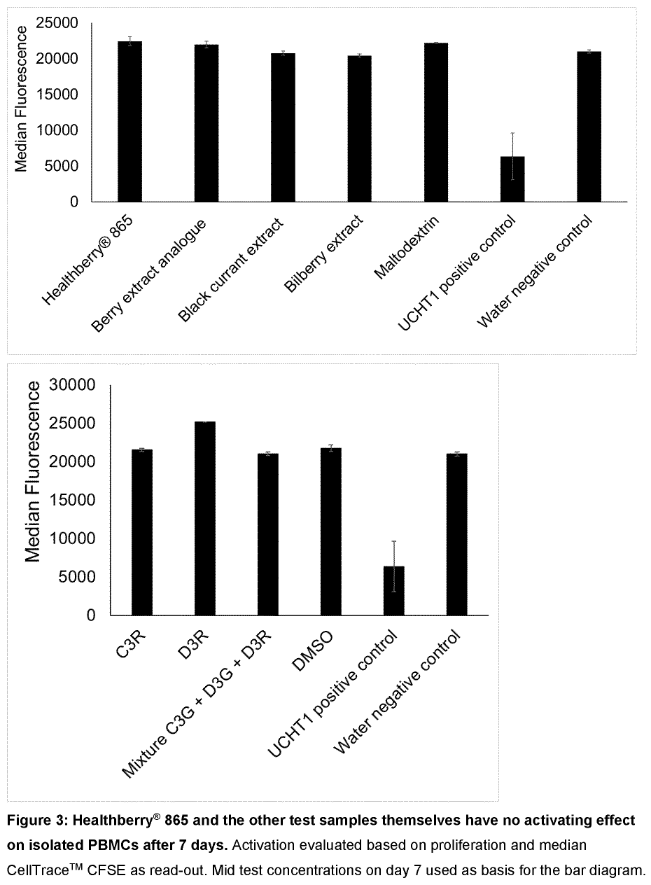

FIG. 3 displays that Healthberry® 865 and the other test samples themselves have no activating effect on isolated PBMCs after 7 days. Activation was evaluated based on proliferation and median CellTrace™ CFSE as read-out. Mid test concentrations on day 7 used as basis for the bar diagram.

In the next experiment the effect of the test compounds on the proliferation of activated PBMCs was evaluated. Therefore, the PBMCs were first activated for 2 days via anti-CD3 (UCHT1) and anti-CD28 activating surface, followed by induction of proliferation with Interleukin-2. These activated PBMCs were then again incubated with the test compounds and the activation of the cells was determined based on cell proliferation as read-out, which was measured via median fluorescence of CellTrace™ CFSE.

TABLE 11
Berry extracts as well as anthocyanins reduce the proliferation of activated PBMCs. Cell
activation evaluated based on proliferation and median CellTrace ™ CFSE as read-
out (the lower the fluorescence the higher the proliferation). Highest test concentrations
should not be considered due to lower viabilities. Average based on assay triplicates.

| | concentration | | | | |
|---|---|---|---|---|---|
| | [μg/mL]; | Median Fluorescence Day 2 | | Median Fluorescence Day 3 | |
| | DMSO & | | standard | | standard |
| | DMF [%] | Average | deviation | Average | deviation |
| Healthberry | 250 | 18079 | 305 | 12292 | 818 |
| | 100 | 15042 | 245 | 6233 | 116 |
| | 50 | 14601 | 127 | 5870 | 120 |
| Berry extract | 150 | 17970 | 485 | 13729 | 1333 |
| analogue | 60 | 14576 | 64 | 6191 | 129 |
| | 30 | 14722 | 353 | 6031 | 100 |
| Black currant | 166.5 | 18655 | 286 | 13747 | 654 |
| extract | 66.6 | 14537 | 104 | 6195 | 194 |
| | 33.3 | 13928 | 265 | 5854 | 77 |
| Bilberry extract | 166.5 | 18475 | 587 | 16057 | 1898 |
| | 66.6 | 14396 | 89 | 6175 | 248 |
| | 33.3 | 14289 | 182 | 5743 | 203 |
| Maltodextrin | 100 | 13932 | 362 | 5320 | 228 |
| | 40 | 14157 | 306 | 5395 | 101 |
| | 20 | 14177 | 83 | 5288 | 48 |
| UCHT1 positive | 5 | 10370 | 174 | 3281 | 30 |
| control | 2 | 10606 | 100 | 3444 | 167 |
| | 1 | 10532 | 91 | 3440 | 71 |
| Water negative control | | 13189 | 157 | 4951 | 114 |
| C3R | 75 | 17032 | 154 | 11086 | 49 |
| | 30 | 15317 | 226 | 7228 | 222 |
| | 15 | 14630 | 322 | 6153 | 264 |
| D3R | 75 | 19716 | 622 | 13927 | n.a. |
| | 30 | 15685 | 132 | 8150 | n.a. |
| | 15 | 14407 | 160 | 6602 | 233 |
| Mixture C3G + | 75 | 17069 | 74 | 11888 | 460 |
| D3G + D3R | 30 | 15307 | 185 | 7376 | 189 |
| | 15 | 14398 | 175 | 6189 | 151 |
| DMSO | 1.5 | 16793 | 236 | 11214 | 272 |
| | 0.6 | 14729 | 622 | 6846 | 263 |
| | 0.3 | 14216 | 479 | 5731 | 170 |
| Astaxanthin | 10 | 19750 | 238 | 15342 | 827 |
| | 4 | 15365 | 204 | 10055 | 120 |
| | 2 | 14190 | 109 | 6502 | 119 |
| DMF | 1.5 | 22952 | 901 | 18547 | 1217 |
| | 0.6 | 17311 | 66 | 11205 | 366 |
| | 0.3 | 15184 | 53 | 6882 | 487 |

| | | | | |
|---|---|---|---|---|
| Water negative control | 13189 | 157 | 4951 | 114 |

After 2 days of incubation no significant difference between the samples could be overserved as expected for the required duration needed to study any influence on immune cell activations. On day 3 the results still displayed almost no difference between the maltodextrin and the water control whereas the UCHT1 (anti-CD3) antibody positive control showed significantly faster proliferation and therefore lower median fluorescence. The increased proliferation of the positive control confirms the activation of the immune cells and in this case also the functionality of the in vitro model.

For DMF and Astaxanthin similar results with slower proliferation were observed, which can be explained by impaired cell viability and secondary effects as the DMF solvent control even resulted in higher fluorescence and therefore lower proliferation than Astaxanthin in DMF.

Furthermore, on day 3 the tested berry extracts including Healthberry® 865 and the berry extract analogue without maltodextrin, displayed a dose-dependent and significant decrease of proliferation in comparison to the maltodextrin and the water control. Incubation with the pure anthocyanins C3R and D3R or a mixture of anthocyanins also showed a dose-dependent and significant decrease of proliferation in comparison to the corresponding DMSO solvent control. Especially the effects on day 3 for the mid concentration of the test compounds clearly illustrate the differences in proliferation.

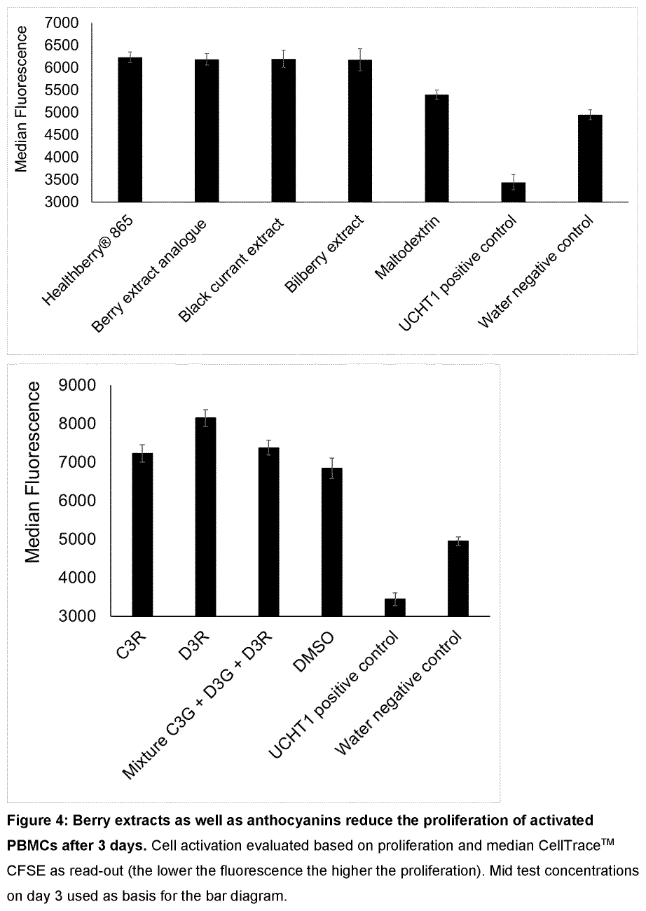

FIG. 4 shows that berry extracts as well as anthocyanins reduce the proliferation of activated PBMCs after 3 days. Cell activation was evaluated based on proliferation and median CellTrace™ CFSE as read-out (the lower the fluorescence the higher the proliferation). Mid test concentrations on day 3 used as basis for the bar diagram.

Considering, that the range between the positive and negative control is represented by ˜1500 fluorescence units as change of the median fluorescence, differences of ˜400 up to ˜1200 between the berry extracts as well as the anthocyanins and their corresponding negative controls confirm the significance of the observed effects. For example, there is a difference in median fluorescence between Healthberry® 865 and the corresponding maltodextrin control of ˜800 fluorescence units which is equivalent to ˜55% of the maximum assay amplitude determined between the water negative control and the UCHT1 positive control.

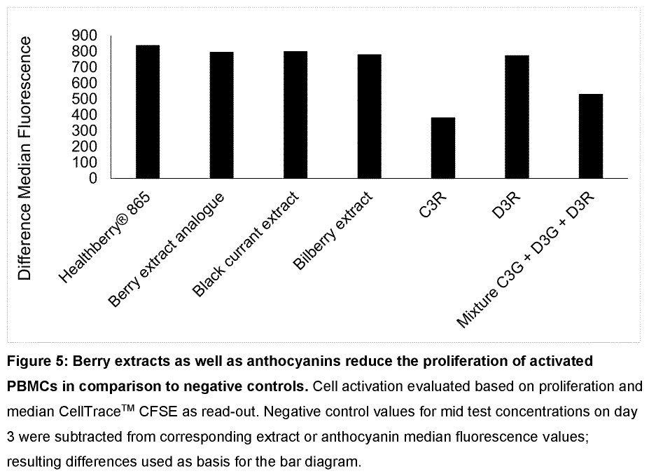

FIG. 5 shows that berry extracts as well as anthocyanins reduce the proliferation of activated PBMCs in comparison to negative controls. Cell activation was evaluated based on proliferation and median CellTrace™ CFSE as read-out. Negative control values for mid test concentrations on day 3 were subtracted from corresponding extract or anthocyanin median fluorescence values; resulting differences used as basis for the bar diagram.

Thus, the cells incubated with berry extracts of black currants and bilberries or with anthocyanins (including mixtures of anthocyanins) proliferate slower than control cells, representing a reduced immune cell activation.

### Example 4: Influence of Berry Extracts and Anthocyanins on T Cell Population and Their Surface Markers (CD4+ & CD8+)

PBMS were activated by using an activating surface and the addition of Interleukin-2 to the cell culture media. The activated PBMCs, which contain T-cells as the most prominent cell type, were treated with the berry extracts or the anthocyanins from day 3 on after isolation. To evaluate the different T cell population flow cytometry analysis were performed with anti-CD4 and anti-CD8 antibodies in combination with Annexin V staining as live-dead-staining. CD4 and CD8 positive cells were displayed in relation to the population of living cells with an overall value reaching almost 100% of the living cells; e.g. Healthberry® 865 reaching a value of 0.32 of CD8+ cells (˜32% of living cells) and 0.64 of CD4+ cells (˜64% of living cells) after 4 days of incubation.

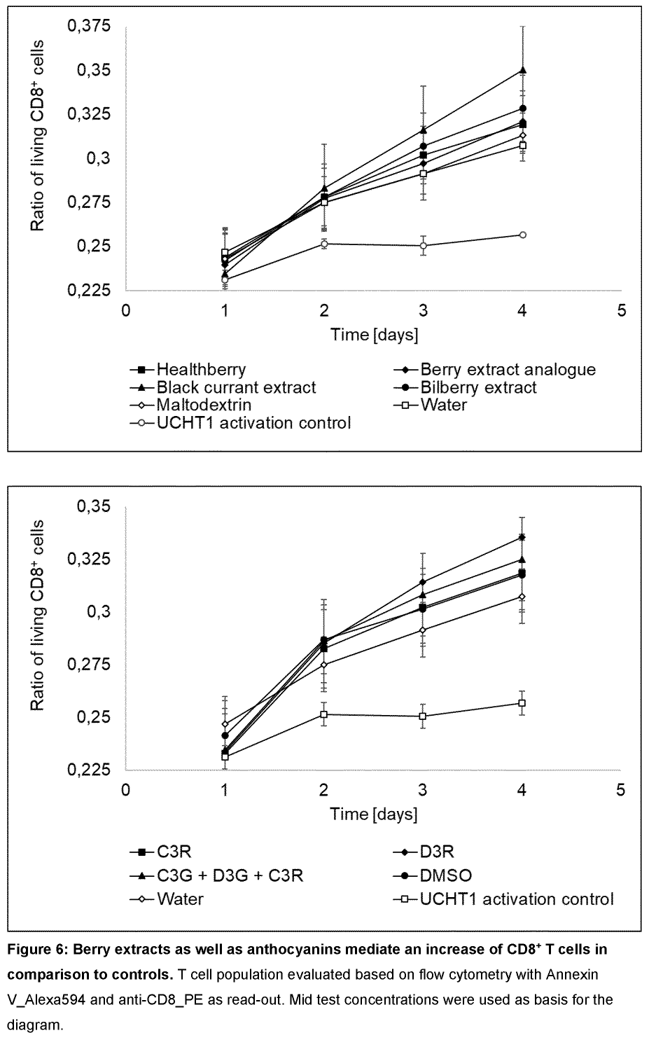

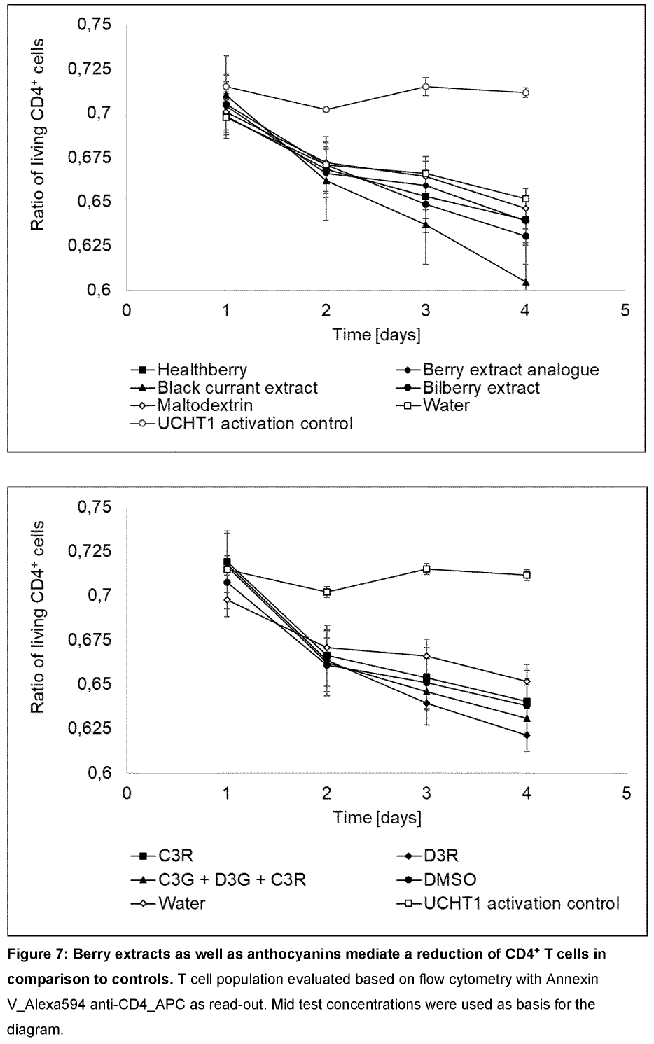

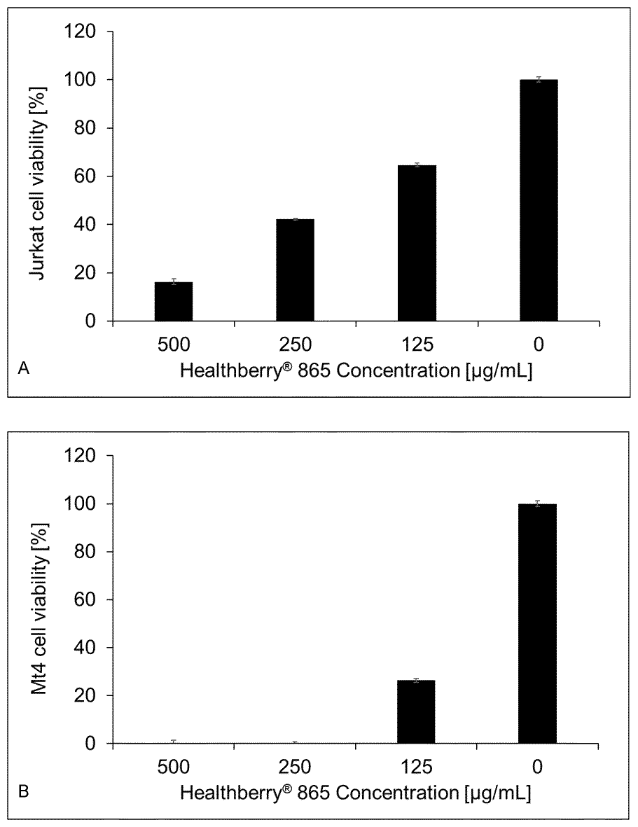

FIG. 6 shows that berry extracts as well as anthocyanins mediate an increase of CD8⁺ T cells in comparison to controls. In contrast FIG. 7 shows that berry extracts as well as anthocyanins mediate a reduction of CD4⁺ T cells in comparison to controls. Especially black currant extract as well as D3R showed the most significant effects, which is in line with the high D3R content present in black currants.

These results confirm that berry extracts and anthocyanins can significantly influence the immune system by the means of modulating the CD-marker profiles and therefore the T cell populations. These effects open a broad range of possible applications such as e.g. CD4⁺-related leukemia.

### Example 5: Specific Influence of Berry Extracts on the Viability of CD4⁺ T-cell Lymphoma Cells

In order to further evaluate the effect of Healthberry® 865 on T cell populations and specifically the CD4+ populations, additional cell viability analysis was performed. On the basis that Healthberry® 865 has an influence on the CD4+ cell populations and additionally on the activation status of T cells, specifically T cell leukemia/lymphoma cell lines as Jurkat (T cell leukemia) and Mt4 (HTLV-I positive; human T-cell lymphotropic virus type I retrovirus causes adult T-cell leukemia-lymphoma) cells were chosen as in vitro models. Furthermore, control cell lines were chosen, which are not CD4⁺ such as HeLa cells (epithelial cervix carcinoma) or which represent another type of leukemia such as THP-1 (acute monocytic leukemia). Surprisingly it was shown that the T cell lymphoma cell line viability of Jurkats or Mt4 cells was significantly reduced by increasing concentrations of Healthberry® 865, whereas the control cell lines HeLa and THP-1 did not display any significant viability reduction. These results open new treatment opportunities of Healthberry® 865 in CD4+ T cell related diseases such as T cell leukemia.

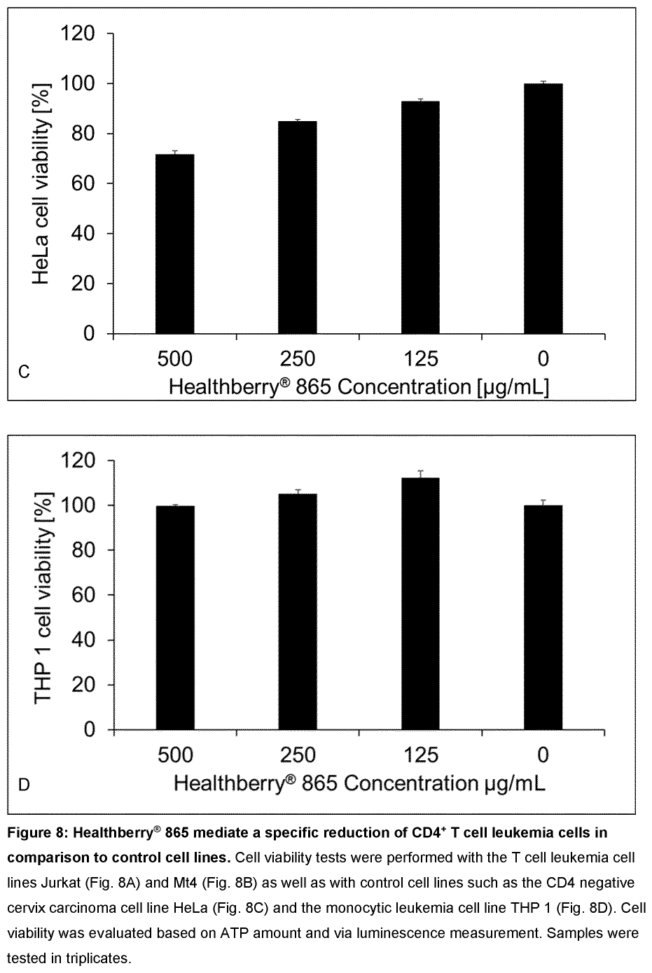

FIG. 8 shows that Healthberry® 865 mediates a specific reduction of CD4⁺ T cell leukemia cells in comparison to control cell lines. Cell viability tests were performed with the T cell leukemia cell lines Jurkat (FIG. 8A) and Mt4 (FIG. 8B) as well as with control cell lines such as the CD4 negative cervix carcinoma cell line HeLa (FIG. 8C) and the monocytic leukemia cell line THP 1 (FIG. 8D). Cell viability was evaluated based on ATP amount and via luminescence measurement. Samples were tested in triplicates.

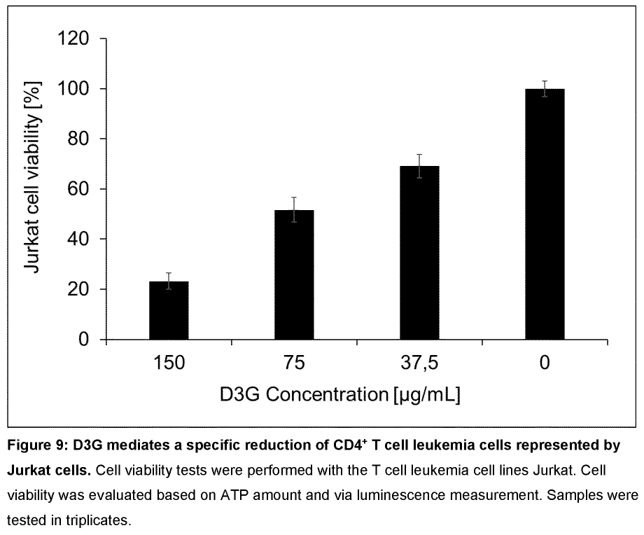

Furthermore, D3G as one of the major single anthocyanins was analyzed as well regarding the influence on the viability of the T cell leukemia cell lines Jurkat. FIG. 9 shows that Delphinidin 3-glucoside/D3G as well mediates a specific reduction of Jurkat cells representing a CD4⁺ T cell leukemia cell line. Cell viability was evaluated based on ATP amount and via luminescence measurement. Samples were tested in triplicates.

1. A method for treating a cancer, the method comprising:
administering a composition to a subject in need thereof,
wherein the cancer comprises cancer cells that are CD4+, and
wherein the composition comprises one or more selected from the group consisting of an extract of black currants, an extract of bilberries, and an anthocyanin.

2. The method of claim 1, wherein the cancer is a T-cell cancer.

3. The method of claim 1, wherein the cancer is a T-cell leukemia or a T cell lymphoma.

4. The method of claim 3, wherein the cancer is Sézary syndrome, Hodgkin's lymphoma, dermatopathic lymphadenitis, or indolent MCL (mantle cell lymphoma).

5. The method of claim 1, wherein a blood sample taken from the subject comprises more than 1200 CD4+ cells/μL.

6. The method of claim 1, wherein a blood sample taken from the subject has a CD4+/CD8+ cell ratio above 2.

7. The method of claim 1, wherein the composition reduces a CD4+/CD8+ cell ratio.

8. The method of claim 1, wherein the composition comprises an extract of black currants, and the black currants are fruit of Ribes nigrum and/or
wherein the composition comprises an extract of bilberries and the bilberries are fruit of Vaccinium myrtilius.

9. The method of claim 1, wherein the composition is an extract of pomaces from black currants and bilberries.

10. The method of claim 1, wherein the composition comprises anthocyanins, and the anthocyanins are present in the composition at a concentration of at least 25 weight-%.

11. The method of claim 1, wherein the extract is prepared by a process comprising the steps of extraction of black currants and/or bilberries, purification via chromatography, mixing of the extract(s) with water and spray-drying of the mixture.

12. The method of claim 1, comprising at least one anthocyanin selected from the group consisting of:
cyanidin-3-glucoside, cyanidin-3-galactoside, cyanidin-3-arabinoside, delphinidin-3-glucoside, delphinidin-3-galactoside, delphinidin -3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, and delphinidin-3-rutinoside.

13. The method of claim 1, wherein the anthocyanin is delphinidin 3-rutinoside.

14. The method of claim 1, further comprising:
treating the subject with an anticancer treatment.

15. The method of claim 1, wherein the composition is administered simultaneously, separately or sequentially with an anticancer treatment.

16. The method of claim 14, wherein the anticancer treatment is chemotherapy, radiotherapy, steroid therapy, targeted therapy, photodynamic therapy, biologic therapy, immunotherapy CAR T therapy, or stem cell therapy.

17. The method of claim 1, wherein the composition further comprises a chemotherapeutic drug.

18. The method of claim 1, wherein the composition comprises anthocyanins and is administered to the subject 1 to 10 oral dosages of at least 80 mg anthocyanins each per day.

19. The method of claim 1, wherein the composition is administered to the subject, reaching a concentration in the target compartment of at least 30 μg/ml.

20. (canceled)

21. A combined preparation comprising:
(i) one or more selected from the group consisting of an extract of black currants, an extract of bilberries, and an anthocyanin; and
(ii) an oral chemotherapeutic drug.

22. The combined preparation of claim 21, wherein the composition is in tablet, capsule or liquid form.

23. The combined preparation according to claim 21, wherein the combined preparation comprises an extract of black currants, and the black currants are fruit of Ribes nigrum,
wherein the composition comprises an extract of bilberries, and the bilberries are fruit of Vaccinium myrtillus, and/or
wherein the composition comprises an anthocyanin, and the anthocyanin is selected from the group consisting of cyanidin-3-glucoside, cyanidin-3-galactoside, cyanidin-3-arabinoside, delphinidin-3-glucoside, delphinidin-3-galactoside, delphinidin -3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, and delphinidin-3-rutinoside.
